(19) United States
(12) Patent Application Publication
(10) Pub. No.: US 2008/0009682 A1
(43) Pub. Date: Jan. 10, 2008

(54) Method and system for clinical interpretation and review of patient data

(76) Inventor: David Hernke, Sussex, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/483,206

(22) Filed: Jul. 7, 2006

(51) Int. Cl. A61B 5/00

(52) U.S. Cl. 600/300

(57) ABSTRACT

The method and system improves the efficiency of the use of trained medical personnel in treating monitored patients. Specifically, this efficiency is improved in remote chronic disease monitoring. The method and system incorporates a pool of call screeners to contact and screen remote patients that have not submitted their data. Depending on the results of the screening, the patient may require assistance from skilled medical personnel and be routed to such personnel. Alternative embodiments may screen all remote patients without first collecting physiological data. A preferred embodiment will include a means for the patient to provide feedback to the caseworker or the skilled medical personnel.

### FIELD OF THE INVENTION

The invention relates to the field of remote patient monitoring. More particularly, the invention relates to the field of chronic disease monitoring.

### BACKGROUND OF THE INVENTION

For a variety of reasons, monitoring of chronically ill patients in a remote, non-hospital environment will become more common in the near future. The clinical data collected, for example, blood pressure, weight, etc., is transmitted back to a caseworker or clinician who can provide early intervention to prevent re-hospitalizations. By monitoring patients remotely, costly re-hospitalization events can be avoided and the overall cost of managing the disease can be reduced.

The clinical monitoring of a physiological state of a patient requires a process flow including first acquiring the desired physiological data via a sensor. Depending upon the design of the particular sensor, this may or may not require trained medical personnel. This clinical monitoring next includes machine collection and interpretation of physiological data. Machine interpretation of collected physiological data is frequently used to “filter” the acquired the physiological data in an attempt to lessen the interaction of trained medical personnel. The machine interpretation may also generate additional clinical data in the form of alarms. The purpose of alarms are to get the attention of clinical personnel so that more immediate action can be taken. The machine collection and interpretation of physiological data does not require trained medical personnel.

Lastly, the clinical monitoring includes clinical interpretation and review of the physiological data. The review may be made on “raw” data or data that has been processed in some manner with a machine based algorithm to aid the medical personnel in making the clinical review. The clinical review of the physiological data requires trained medical personnel. The end outcome of the clinical review is a clinical diagnosis and/or clinical decision. In current practice, trained medical personnel are only necessarily required for the clinical interpretation and review step, but are frequently used in the entire process. As a result, trained medical personnel are inefficiently utilized, and coupled with a shortage of trained medical personnel, this impacts the ability to clinically monitor every patient requiring attention from trained medical personnel.

### SUMMARY OF THE INVENTION

The method and system improves the efficiency of the use of trained medical personnel in treating monitored patients. Specifically, this efficiency is improved in remote chronic disease monitoring. The method and system incorporates a pool of call screeners to contact and screen remote patients that have not submitted their data. Depending on the results of the screening, the patient may require assistance from skilled medical personnel and be routed to such personnel. Alternative embodiments may screen all remote patients without first collecting physiological data. A preferred embodiment will include a means for the patient to provide feedback to the caseworker or the skilled medical personnel.

One aspect of the present invention is a method of clinically interpreting and reviewing patient data, the method comprises collecting a set of physiological data from each of a plurality of patients, triaging the set of physiological data from each of the plurality of patients to determine whether any of the plurality of patients need clinical attention and whether the set of physiological data for any of the plurality of patients has not been collected, and screening those of the plurality of patients that did not have the set of physiological data collected, and forwarding a set of screening data to a skilled medical personnel when the screening data indicates that one of the plurality of patients needs clinical attention. The collecting step includes sensing the set of physiological data with a remote sensing system, and each of the plurality of patients submitting the set of physiological data sensed by the remote sensing system. The screening step includes a screener contacting the plurality of patients that did not have the set of physiological data collected and further wherein the screener submitting a set of scripted questions to those patients that did not have the set of physiological data collected, the screening data includes a set of answers to the set of scripted questions and the screener assisting one of the plurality of patients when the screening data indicates that one of the plurality of patients does not need medical attention. When the triaging step determines the physiological data for one of the plurality of patients has not been collected, a blank record corresponding to the patient is placed in a queue wherein a plurality of screeners select from a plurality of blank records in the queue which is located on a secure internet site or may be located in a database. The method of the present invention further comprises any one of the plurality of patients providing a set of feedback corresponding to a perceived level of care.

Another aspect of the present invention is a system for clinically interpreting and reviewing patient data, the system comprises a monitoring system configured to collect a set of physiological data from a plurality of patients, a storage media for storing a computer application, and a processor coupled to the monitoring system and the storage media, the processor configured to execute the computer application, and further configured to receive the set of physiological data from the monitoring system, wherein when the computer application is executed, the set of physiological data is triaged to determine whether any of the plurality of patients need clinical attention and whether the set of physiological data for any of the plurality of patients has not been collected, and further wherein a patient record is placed in a queue for each of the plurality of patients that did not have the set of physiological data collected, such that a plurality of screeners may select a patient record from the queue to perform a screening. The plurality of screeners forwards a set of screening data resulting from the screening, wherein each of the plurality of patients submits to the processor the set of physiological data collected by the remote sensing system. The screening includes one of the plurality of screeners contacting the plurality of patients that did not have the set of physiological data collected wherein the screening includes the screener submitting a set of scripted questions to the plurality of patients that did not have the set of physiological data collected, and further wherein the screening data includes a set of answers to the set of scripted questions and the screener assists one of the plurality of patients when the screening data indicates that one of the plurality of patients does not need medical attention. The system is configured to receive a set of feedbacks corresponding to a perceived level of care from any of the plurality of patients and further comprises a network configured to couple the plurality of screeners to the processor.

Yet another aspect of the present invention is a method of clinically interpreting and reviewing patient data, the method comprises collecting a set of physiological data from each of a plurality of patients, screening the plurality of patients, and forwarding a set of screening data to a skilled medical personnel when the screening data indicates that one of the plurality of patients needs clinical attention. The collecting step includes sensing the set of physiological data with a remote sensing system, and each of the plurality of patients submitting the set of physiological data sensed by the remote sensing system. The screening step includes the screener submitting a set of scripted questions to the plurality of patients and further wherein the screening data includes a set of answers to the set of scripted questions, the screener assisting one of the plurality of patients when the screening data indicates that one of the plurality of patients does not need medical attention and further comprises any one of the plurality of patients providing a set of feedback corresponding to a perceived level of care.

### BRIEF DESCRIPTION OF THE DRAWINGS

FIGS. 1a and 1b illustrate flow charts of a method in accordance with embodiments of the present invention.

FIG. 2 illustrates a graphical representation of a system in accordance with an embodiment of the present invention.

FIG. 3 illustrates a block diagram of a system in accordance with an embodiment of the present invention.

### DETAILED DESCRIPTION OF THE INVENTION

This invention recognizes that acquiring physiological data does not in all cases imply that the patient is located in the same physical facility as the trained medical personnel. In fact, a preferred embodiment contemplates a patient being monitored from his or her home. This invention also recognizes that the machine collection and interpretation of physiological data does not need to be located in the same physical facility as the trained medical personnel or the patient. For example, a remotely located “data-center” may collect the physiological data into an electronic medical record database.

This invention addresses the method used to clinically interpret and review the physiological data. It is common clinical practice to assign a large number of patients to a specific caseworker within a common physical facility with the other caseworkers. For example, there may be 10,000 patients in a particular account. On any particular day, physiological readings may come back from 9,000 of the patients. That data is run through a triage algorithm and finds 50 of the 9,000 patients may require clinical intervention. Those 50 patient issues are forwarded on to a highly-skilled clinical person for follow-up. However, one thousand of the patients didn't submit their data on this day, where the submission is through a home monitoring device or an IVR system. Some of the patients have merely forgotten, while others require some coaching, and still some others may be depressed or generally non-compliant. In order to make sure all of the patients are accounted for, 1,000 phone calls need to be made.

In one embodiment, these 1,000 calls can be thought of as non-clinical tasks and can be placed on a secure internet site that allows a pool of trained screeners to select any number of these tasks. Some may choose many tasks, other just a few, depending on their personal schedules. The screeners may be compensated based on how many tasks they complete. As the screener's shift ends, the compensation rate may rise to encourage all calls to be made. The screeners contact the patient by making a telephone call and using a script, e.g., “what do you weigh?”, “are you having shortness of breath?”, “are you depressed?”, and many of the likely issues can be resolved by the screener, e.g., “I forgot to weigh myself”, “I forgot how to turn the device on”, “I was too tired.” Depending on the responses from the scripted questions, the screener can escalate an issue to a clinical care provider such as a caseworker, doctor, or emergency service—911.

Another embodiment could be implemented in a traditional disease management approach. In the traditional approach, caseworkers, sometimes called health coaches, contact the patients at regular intervals that vary according to many factors. For example, caseworkers may contact patients at weekly intervals to collect physiological data such as blood pressure and weight. They may contact patients at monthly intervals to provide clinical education and positive reinforcement, e.g., what is a low sodium diet? methods to quit smoking. Those phone calls can be treated as tasks which could be performed by low skilled workers located in the US or in low cost countries. These tasks can be placed on a secure internet site for disbursement to the pool of low skilled workers. These workers would ask scripted questions and record responses on a website. If an answer to a scripted question indicates intervention is required, the issue is forwarded to a higher skilled clinical person for resolution.

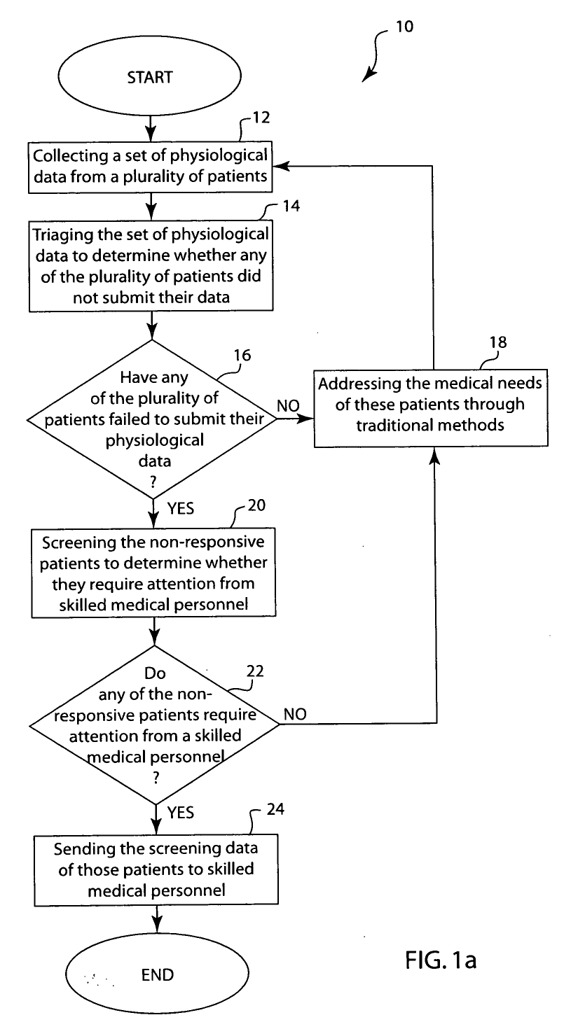

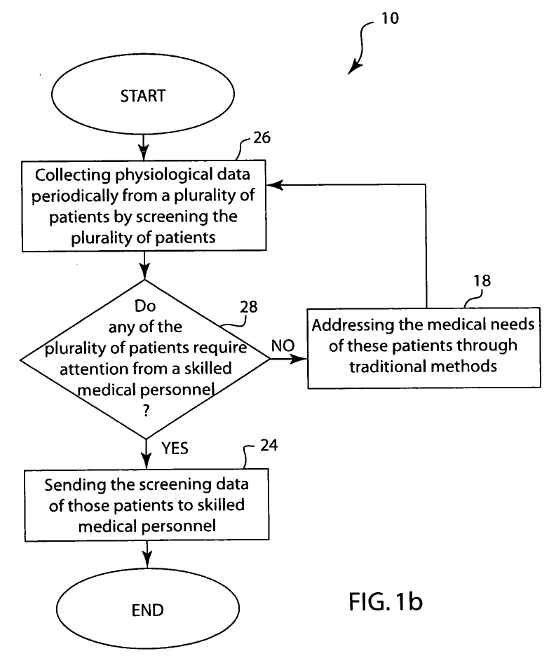

Referring to FIG. 1a, a review method 10 of the preferred embodiment is depicted. In step 12, a set of physiological data is collected from a plurality of remote patients. As stated above, these remote patients are monitored preferably in their home environment for a number of physiological parameters. While some remote monitoring systems are configured to automatically collect and send the set of physiological data from these patients, some remote sensing systems require the assistance and cooperation of the patient to properly and accurately collect and relay all of the information to the physician or caregiver. In step 14, the set of physiological data that is collected from each of the plurality of patients is triaged to determine whether any of the plurality of patients did not submit their data. In other words, it will be determined in step 14 whether any of the patients did not cooperate or may be having difficulties collecting and relaying their personal physiological data. In step 16, if all of the plurality of patients successfully relayed their data, then in step 18, the medical needs of these patients are addressed through traditional methods. In other words, the particular caregiver system that is implemented will determine whether each patient requires the care of a caseworker, nurse, physician or emergency personnel.

Still referring to FIG. 1a, if in step 16, it is found that the triaging step in 14 has uncovered any of the plurality of patients that have failed to submit their physiological data, then in step 20, the non-responsive patients are screened to determined whether they require attention from skilled medical personnel. These non-responsive patients include both patients that have been uncooperative, or have had difficulties in collecting and relaying their physiological data. Referring back to step 20, the screening step is carried out preferably by a group of unskilled callers that will contact the non-responsive patients and go through a scripted questionnaire with the non-responsive patient to determine whether any of the non-responsive patients require attention from a skilled medical professional in step 22. The screening step 20 will be effectuated by each of the unskilled callers picking the records of the non-responsive patients from a queue, where such queue can be configured on a secure internet website or in a database, or in any other manner known in the art. As described above, there may be various incentives configured for the screeners to be productive.

Still referring to FIG. 1a, in step 22, if none of the non-responsive patients require attention from skilled medical personnel, then the needs of these non-responsive patients are addressed through traditional methods in step 18, and the review method 10 begins again in step 12. However, if any of the non-responsive patients require attention from skilled medical personnel in step 22, then in step 24, the screening data obtained through the scripted questionnaire in the screening step 20 is sent to the skilled medical personnel so that the non-responsive patient receives attention.

An alternative embodiment of the review method 10 is depicted in FIG. 1b. Here, in step 26, a set of physiological data is periodically collected from the plurality of patients by screening the plurality of patients as was described in step 20 of FIG. 1a. In this alternative embodiment, instead of collecting the set of physiological data and triaging the data to see whether there are any non-responsive patients, the physiological data is merely collected periodically from each remote patient, providing a more monitored system, but a system that requires additional screeners. Referring back to FIG. 1b, in step 28, if none of the plurality of patients require attention from skilled medical personnel, then the medical needs of these patients are addressed through traditional methods in step 18 and the review method 10 begins again in step 26. However if any of the plurality of patients require attention from skilled medical personnel in step 28, then in step 24, the screening data obtained in step 26 is sent to skilled medical personnel so that the patients requiring this higher level of care are treated accordingly.

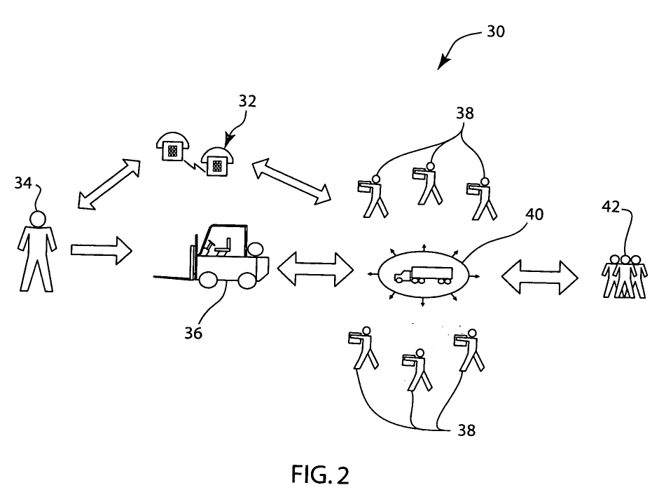

FIG. 2 depicts a graphical representation of a review system 30. In this review system 30, the remote patient 34 submits a set of physiological data to a database 36. If any of the remote patients 34 being monitored by the review system 30 do not submit a set of physiological data, then a record for this patient 34 is placed in a queue that may be located in a secure portion of a network 40, or in the database 36.

Still referring to FIG. 2, a number of caseworkers 38, preferably unskilled call screeners, may select patient 34 records from the queue and contact the patient 34 via a communication system 32 in order to complete a scripted questionnaire with the patient 34. If the caseworker 38, through the scripted questionnaire receives screening data that indicates that the patient 34 requires skilled medical personnel 42, then the caseworker 38 relays this screening data onto the skilled medical personnel 42 so that the patient 34 may receive the appropriate care. Once again, it is contemplated that the caseworker 38 may be unskilled callers simply reading a scripted questionnaire, but it is also contemplated that the caseworker 38 may be trained to some degree medically. Furthermore, the communication system 32 is preferably a simple telephone line, but may also be implemented as an internet chat connection, assisted website connection or any communication system 32 that would allow the caseworker 38 to complete a questionnaire with the patient.

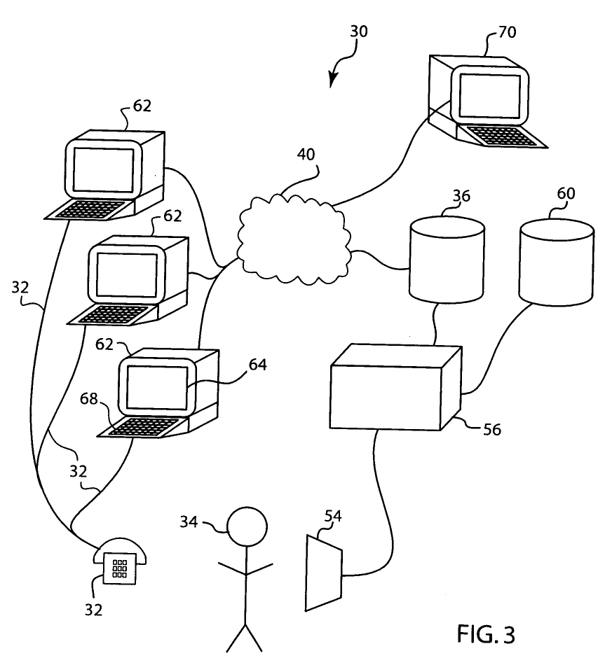

The review system 30 is also depicted in the block diagram in FIG. 3. In FIG. 3, the patient 34 submits physiological data through a monitoring system 54 and the monitoring system 54 sends the physiological data to a processor 56. A storage media 60 stores a set of computer code that when executed by the processor 56 carries out the triaging step and the remainder of the method discussed in FIGS. 1a and 1b. Records for the non-responsive patients 34 are stored in a queue as stated previously either in the database 36 or on a secure website on the network 40, wherein such network may be the internet or any land or network 40 known in the art. Caseworkers may utilize a caseworker workstation 62 with a graphical user interface 64 and an input device 68 in order to select patient 34 records from the queue in the network 40 or database 36, and contact those patients 34 through a communication system 32 in order to complete the scripted questionnaire with the patient 34. The review system 30 also includes a medical workstation 70, where skilled medical personnel can receive through the network 40 the screening data obtained from those patients 34 that require immediate medical assistance from skilled medical personnel. In further embodiments, the skilled medical personnel may contact the patient 34 through the medical workstations 70, or through the communication system 32. This configuration however is not depicted in FIG. 3.

In the preferred embodiment the patient could also be allowed to provide electronic feedback on the level of care he/she received. This feedback could be used as a method for reviewing the quality of service being provided. Referring to FIG. 3, the patient 34 could provide feedback to the screener through the screening workstation 62 and the communication system 32, or directly to the physician at the medical workstation 70 through communication system 32 and network 40, or by a patient workstation (not shown).

The system and method as described allows a more efficient utilization of skilled medical personnel. This in turn allows the expansion of the number of patients that can be monitored at any one time. The system and method as described utilizes the internet and any other network known in the art as a communication media for accessing electronic medical records by medical caseworkers for the purpose of triaging a large amount of patient data and the disposition of the review data either for archive or further review, and allows for the more efficient use of skilled nursing staff.

The present invention has been described in terms specific embodiments incorporating details to facilitate the understanding the principles of construction and operation of the invention. Such reference herein to specific embodiments and details thereof is not intended to limit scope of the claims appended hereto. It will be apparent to those skilled in the art that modifications may be made in the embodiment chosen for illustration without departing from the spirited scope of the invention.

We claim:

1. A method of clinically interpreting and reviewing patient data, the method comprising:
collecting a set of physiological data from each of a plurality of patients;
triaging the set of physiological data from each of the plurality of patients to determine whether any of the plurality of patients need clinical attention and whether the set of physiological data for any of the plurality of patients has not been collected; and
screening those of the plurality of patients that did not have the set of physiological data collected; and
forwarding a set of screening data to a skilled medical personnel when the screening data indicates that one of the plurality of patients needs clinical attention.

2. The method as claimed in claim 1, wherein the collecting step includes sensing the set of physiological data with a remote sensing system, and each of the plurality of patients submitting the set of physiological data sensed by the remote sensing system.

3. The method as claimed in claim 1, wherein the screening step includes a screener contacting the plurality of patients that did not have the set of physiological data collected.

4. The method as claimed in claim 3, wherein the screening step includes the screener submitting a set of scripted questions to the plurality of patients that did not have the set of physiological data collected, and further wherein the screening data includes a set of answers to the set of scripted questions.

5. The method as claimed in claim 3, further comprising the screener assisting one of the plurality of patients when the screening data indicates that one of the plurality of patients does not need medical attention.

6. The method as claimed in claim 1, wherein when the triaging step determines the physiological data for one of the plurality of patients has not been collected, a blank record corresponding to the patient is placed in a queue.

7. The method as claimed in claim 6, wherein a plurality of screeners select from a plurality of blank records in the queue.

8. The method as claimed in claim 6, wherein the queue is located on a secure internet site.

9. The method as claimed in claim 6, wherein the queue is located in a database.

10. The method as claimed in claim 1, further comprising any one of the plurality of patients providing a set of feedback corresponding to a perceived level of care.

11. A system for clinically interpreting and reviewing patient data, the system comprising:
a monitoring system configured to collect a set of physiological data from a plurality of patients;
a storage media for storing a computer application; and
a processor coupled to the monitoring system and the storage media, the processor configured to execute the computer application, and further configured to receive the set of physiological data from the monitoring system,
wherein when the computer application is executed, the set of physiological data is triaged to determine whether any of the plurality of patients need clinical attention and whether the set of physiological data for any of the plurality of patients has not been collected, and further wherein a patient record is placed in a queue for each of the plurality of patients that did not have the set of physiological data collected, such that a plurality of screeners may select a patient record from the queue to perform a screening.

12. The system as claimed in claim 11, wherein the plurality of screeners forwards a set of screening data resulting from the screening.

13. The system as claimed in claim 11, wherein each of the plurality of patients submits to the processor the set of physiological data collected by the remote sensing system.

14. The system as claimed in claim 11, wherein the screening includes one of the plurality of screeners contacting the plurality of patients that did not have the set of physiological data collected.

15. The system as claimed in claim 14, wherein the screening includes the screener submitting a set of scripted questions to the plurality of patients that did not have the set of physiological data collected, and further wherein the screening data includes a set of answers to the set of scripted questions.

16. The system as claimed in claim 14 wherein the screener assists one of the plurality of patients when the screening data indicates that one of the plurality of patients does not need medical attention.

17. The system as claimed in claim 11, wherein the queue is located on a secure internet site.

18. The system as claimed in claim 11, wherein the queue is located in a database.

19. The system as claimed in claim 11, wherein the system is configured to receive a set of feedbacks corresponding to a perceived level of care from any of the plurality of patients.

20. The system as claimed in claim 11, further comprising a network configured to couple the plurality of screeners to the processor.

21. A method of clinically interpreting and reviewing patient data, the method comprising:
collecting a set of physiological data from each of a plurality of patients;
screening the plurality of patients; and
forwarding a set of screening data to a skilled medical personnel when the screening data indicates that one of the plurality of patients needs clinical attention.

22. The method as claimed in claim 21, wherein the collecting step includes sensing the set of physiological data with a remote sensing system, and each of the plurality of patients submitting the set of physiological data sensed by the remote sensing system.

23. The method as claimed in claim 21, wherein the screening step includes the screener submitting a set of scripted questions to the plurality of patients and further wherein the screening data includes a set of answers to the set of scripted questions.

24. The method as claimed in claim 21, further comprising the screener assisting one of the plurality of patients when the screening data indicates that one of the plurality of patients does not need medical attention.

25. The method as claimed in claim 21, further comprising any one of the plurality of patients providing a set of feedback corresponding to a perceived level of care.
